(19) United States
(12) Patent Application Publication
(10) Pub. No.: US 2018/0193378 A1
(43) Pub. Date: Jul. 12, 2018

(54) BATHWATER AND SOAK ADDITIVE

(71) Applicants: Paul Morris, Flint (GB); Wayne Walton, Flint (GB)

(72) Inventors: Paul Morris, Flint (GB); Wayne Walton, Flint (GB)

(21) Appl. No.: 15/399,784

(22) Filed: Jan. 6, 2017

(51) Int. Cl. A61K 31/785; A61K 33/14

(52) U.S. Cl. CPC A61K 31/785; A61K 33/14

(57) ABSTRACT

The present disclosure generally relates to a method of creating a bathwater soak which renders bathwater viscous and slippery.

### CROSS REFERENCE TO RELATED APPLICATIONS

This application is a continuation in part of application Ser. No. 14/564,349, filed Dec. 9, 2014, and incorporated herein by reference.

### BACKGROUND OF THE INVENTION

### Field of the Invention

The present invention generally relates to a bath additive. Specifically, the present invention relates to an amusing bath additive, particularly for children and/or for therapeutic and spa-like soaks. More particularly, the present invention relates to a mixture that, when added to bathwater, turns the bathwater viscous and transforms the bath into a play area and also into a more soothing solution for those possibly having skin ailments.

### Description of the Related Art

There have been many products in the past for inducing children to take baths when they otherwise decline to do so. Parents have long added detergents or other froth inducing substances to make a child's bath more alluring. These have been met with varying degrees of success.

There have also been many bath additives for skin softening and other skin therapeutic treatments. For example, oils and other emollients have been added to bathwater for some time. These have also been met with varying degrees of success.

Accordingly, there is now provided with this invention an improved bath additive effectively overcoming the aforementioned difficulties and longstanding problems inherent in coaxing children to take baths as well as providing a soothing bath for those in such need.

### SUMMARY OF THE INVENTION

According to one aspect of certain embodiments of the invention, a bathwater additive is disclosed comprising a mixture of salt and acrylamide/sodium acrylate copolymer in the range from about 5% to about 75% by weight, preferably from about 5% to 49% by weight. When the mixture is added to the bathwater a viscous, slippery consistency is created. As used herein, salt refers to common salt, i.e., sodium chloride salt.

According to another aspect of an embodiment of the invention, a method of creating a bathwater additive is disclosed. The bathwater additive comprises mixing salt and acrylamide/sodium acrylate copolymer in the range from about 5% to about 75% by weight, preferably from about 5% to 49% by weight. When the mixture is added to the bathwater a viscous, slippery consistency is created.

Certain embodiments of the present invention comprise a method of creating a viscous bathwater soak. In these embodiments, the method may include the steps of (a) creating a mixture by combining sodium chloride salt and acrylamide/sodium acrylate copolymer in the range from about 5% to about 49% of the salt by weight, and (b) combining the mixture with bathwater to create a viscous bathwater soak.

Such methods may include in step (a) the step of combining a dye in the range of about 1% by weight, and/or combining a fragrance in the range of about 1% by weight, and/or combining an oil in the range from about 5% to about 95% or about 10% to about 60% by weight, and/or combining bicarbonate of soda in the range from about 1% to about 40% by weight, and/or combining citric acid in the range from about 1% to about 10% by weigh, and/or combining vitamins in the range from about 0.1% to about 5% by weight, and/or combining magnesium silicate in the range from about 0.1% to about 5% by weight. Any combination of the foregoing additives may also be used.

The foregoing Summary of the Invention is not intended to limit the scope of the disclosure contained herein nor limit the scope of the appended claims. To the contrary, as will be appreciated by those persons skilled in the art, variations of the foregoing described embodiments may be implemented without departing from the claimed invention.

### DESCRIPTION OF THE PREFERRED EMBODIMENT

The following formulation of a mixture is created for adding to bathwater and/or soak (collectively referred to as “bathwater” herein) for creating a viscous consistency that is both alluring and amusing to children and one that provides relief to those with a wide variety of skin ailments.

The formulation of the bathwater additive mixture is created by adding salt, provided by, for example, Morton Salt, Dupont, or any other salt supplier to acrylamide/sodium acrylate copolymer, provided by, for example, Dupont, BASF, SNF, or any other chemical supplier. The salt is added to the acrylamide/sodium acrylate copolymer in the range of from about 5% to about 75% by weight, preferably from about 5% to 49% by weight. In other embodiments, the salt is added to the acrylamide/sodium acrylate copolymer in the range of from about 20% to about 49% by weight. It has been found that the addition of salt is important for creating this mixture and without it, although the water would thicken, it would not become properly viscous, stringy and/or malleable and thus not form the bathwater soak disclosed herein.

Traditionally, acrylamide/sodium acrylate copolymer is used in the printing industry, however in a more concentrated form than used herein, as in an industrial grade. The present acrylamide/sodium acrylate copolymer is in a form that is sufficiently pure and dilute as to render it safe to apply on skin.

Unlike other bath additives, the present formulation is not a supra absorbent powder which absorbs the water into each bead which then swells and binds the powder and the water together at a molecular level. Instead, in the present formulation, the less water, the stronger these bonds are and the thicker the powder. Hence when you add more water, the bonds weaken until they break, removing the viscous state and returning the character of the bathwater to that of ordinary water.

Also different from other bath additives, the consistency of the present formulation is different, that is, it is smooth, not beads. Another difference between this and other bath additives is that there is no residue after use.

Although the above-described formulation of the mixture may be applied directly to the bathwater, other formulations with additions to this are also described herein. The additives may used purely to provide the end user with a slightly different outcome, for example, a red color instead of a blue color, or a strawberry scented product rather than vanilla.

An additive to the above-described formulation of the mixture may include a dye or a fragrance. Such dyes or fragrances may be added to the above-described formulation in the range of about 1% by weight.

A wide variety of oils may also be added to the mixture. Such oils may be included in the range from about 5% to about 95% by weight. Optimally, the oils may be included in the range from about 10% to about 60% by weight and are particularly advantageous for those seeking a soothing bath.

Another formulation of the mixture may contain bicarbonate of soda. Such a mixture is particularly advantageous for a child's bath. The bicarbonate of soda may be included in the range from about 1% to about 40% by weight.

A further formulation of the mixture may contain citric acid. Such a mixture is particularly advantageous for a child's bath. The citric acid may be included in the range from about 1% to about 10% by weight.

A still further formulation of the mixture may contain one or more vitamins. The vitamins may be included in the range from about 0.1% to about 5% by weight and are particularly advantageous for those seeking a soothing bath or those needing additional vitamin supplements.

Yet another formulation of the mixture may contain talcum powder which may be used as, for example, a skin softener. The talcum powder may be included in the range from about 5% to about 40% by weight.

Another formulation of the mixture may contain magnesium silicate which may be particularly advantageous for those seeking a soothing bath. The magnesium silicate may be included in the range from about 0.1% to about 5% by weight.

Still other formulations may include any or all combinations of dyes, fragrances, oils, bicarbonate of soda, citric acid, vitamins, talcum powder, and magnesium silicate which may be included in the range from about 0% to about 40% by weight. Still other additives may occur to others skilled in the art.

Although the particular embodiments shown and described above will prove to be useful in many applications in the art to which the present invention pertains, further modifications of the present invention will occur to persons skilled in the art. All such modifications are deemed to be within the scope and spirit of the present invention as defined by the appended claims.

What is claimed is:

1. A method of creating a viscous bathwater soak, comprising the steps of:
(a) creating a mixture by combining sodium chloride salt and acrylamide/sodium acrylate copolymer in the range from about 5% to about 49% of the salt by weight;
(b) combining the mixture with bathwater to create the viscous bathwater soak.

2. The method of claim 1, wherein the step of creating a mixture further comprises the step of combining a dye in the range of about 1% by weight.

3. The method of claim 1, wherein the step of creating a mixture further comprises the step of combining a fragrance in the range of about 1% by weight.

4. The method of claim 1, wherein the step of creating a mixture further comprises the step of combining an oil in the range from about 5% to about 95% by weight.

5. The method of claim 4, wherein the oil is in the range from about 10% to about 60% by weight.

6. The method of claim 1, wherein the step of creating a mixture further comprises the step of combining bicarbonate of soda in the range from about 1% to about 40% by weight.

7. The method of claim 1, wherein the step of creating a mixture further comprises the step of combining citric acid in the range from about 1% to about 10% by weight.

8. The method of claim 1, wherein the step of creating a mixture further comprises the step of combining vitamins in the range from about 0.1% to about 5% by weight.

9. The method of claim 1, wherein the step of creating a mixture further comprises the step of combining magnesium silicate in the range from about 0.1% to about 5% by weight.

10. The method of claim 1, wherein the step of creating a mixture further comprises the step of combining any one or more selected from the group comprising dyes, fragrances, oils, bicarbonate of soda, citric acid, vitamins, and magnesium silicate.

11. A method of creating a viscous bathwater soak, comprising the steps of:
(a) creating a mixture by combining sodium chloride salt and acrylamide/sodium acrylate copolymer in the range from about 5% to about 49% of the salt by weight;
(b) combining the mixture with bathwater to create the viscous bathwater soak, wherein said viscous bathwater soak is comprised of substantially non-swollen acrylamide/sodium acrylate copolymer.

12. The method of claim 11, wherein the step of creating a mixture further comprises the step of combining a dye in the range of about 1% by weight.

13. The method of claim 11, wherein the step of creating a mixture further comprises the step of combining a fragrance in the range of about 1% by weight.

14. The method of claim 11, wherein the step of creating a mixture further comprises the step of combining an oil in the range from about 5% to about 95% by weight.

15. The method of claim 14, wherein the oil is in the range from about 10% to about 60% by weight.

16. The method of claim 11, wherein the step of creating a mixture further comprises the step of combining bicarbonate of soda in the range from about 1% to about 40% by weight.

17. The method of claim 11, wherein the step of creating a mixture further comprises the step of combining citric acid in the range from about 1% to about 10% by weight.

18. The method of claim 11, wherein the step of creating a mixture further comprises the step of combining vitamins in the range from about 0.1% to about 5% by weight.

19. The method of claim 11, wherein the step of creating a mixture further comprises the step of combining magnesium silicate in the range from about 0.1% to about 5% by weight.

20. The method of claim 11, wherein the step of creating a mixture further comprises the step of combining any one or more selected from the group comprising dyes, fragrances, oils, bicarbonate of soda, citric acid, vitamins, and magnesium silicate.
